(19) United States
(12) Patent Application Publication
(10) Pub. No.: US 2004/0243038 A1
(43) Pub. Date: Dec. 2, 2004

(54) Walking aid

(76) Inventor: Noble Patterson, Tyrone (GB)

Correspondence Address: DRINKER BIDDLE & REATH, ONE LOGAN SQUARE, 18TH AND CHERRY STREETS, PHILADELPHIA, PA, 19103-6996, US

(21) Appl. No.: 10/486,015

(22) PCT Filed: Aug. 6, 2002
(86) PCT No.: PCT/GB02/03627

(30) Foreign Application Priority Data
Aug. 6, 2001 (GB) 0119104.8

(51) Int. Cl. A61F 5/00

(52) U.S. Cl. 602/23

(57) ABSTRACT

The present invention relates to a walking aid attachable to a human or animal leg frame, used for broken or fractured bones or leg deformities, wherein the aid includes at least one weight bearing actuator adaptable to take some or all of the user's weight when walking. Suitably two actuators are utilized, each consisting of a gas strut.

The present invention concerns a walking aid that is attachable to leg frames used to help leg problems such as broken or fractured bones or leg deformities in human or animal legs to heal and be correct, by assisting “early walking”.

Currently, when patients have traumatic leg injuries such as large or multiple breaks in the major bones of the lower leg, a frame, such as an Ilizarov frame or the American Taylor Spatal frame, is fixedly attached around and through the lower leg using pins that pierce the skin and relevant bones. This support provides a stable frame around the lower leg, and aligns the bone fragments correctly so that they may in time reattach to re-form into a single bone structure. Such lower leg frames also aid walking by the patient as soon as possible after the frame has been fixed to the injured leg.

One of the most important aspects of bone healing is that the bone cells (chondrocytes and osteocytes) involved in producing and developing new bone tissue, respond to pressure on the bone, e.g. by walking, to help them grow.

However, often one of the greatest obstacles for the patient to overcome is the mental block in using the framed leg to walk. The patient must also gauge how much pressure he or she can apply to the injured leg without causing excess pain and discomfort. Unfortunately, this often leads to ineffective use of the frame and possibly prolonged and unnecessarily extended recovery periods for the patient. This is particularly exacerbated when the patient has a broken or severely damaged ankle as well.

Thus, according to one aspect of the present invention there is provided a walking aid attachable to a human or animal leg frame, used for broken or fractured bones, or leg deformities, wherein the aid comprises one or more weight-bearing actuators adapted to take some or all of the user's weight when walking.

The walking aid, attached to the leg frame, is used by patients to help them walk, which in turn facilitates rapid recovery and re-growth of the leg bone by improving mobility. It also reduces dependency on wheelchairs, time and effort in hospital, and overall recovery time.

In addition, certain leg or other orthopaedic deformities or problems are currently improved or assisted with using similar leg frames. Early walking with these could also be provided by the present invention.

Preferably, the walking aid uses two or more actuators.

Preferably the actuator(s) are piston or ram and housing arrangements, such as gas struts, and they are permanently or separably fixable to the outside of the leg frame. The actuators are preferably positioned substantially symmetrically around the frame.

The actuator(s) are preferably dampened and/or have another controlled extension/retraction action.

Preferably the gas strut is a hydraulic gas strut.

Preferably the walking aid is adjustable to accommodate the weight of differently sized patients. The weight bearing capacity of the walking aid could also be adjusted to get the desired amount of pressure on the injured foot or ankle by applying the required balance of upward pressure to the frame.

The walking aid preferably further include flexible fittings and/or mountings such as rubber mountings, rose joints, intermediate swivel means, as well as swivel bases, possibly involving universal joint means, to assist or allow twisting motion of the leg that normally occurs during walking.

Between the patient's foot and the walking aid, there may also be a connecting sleeve that connects onto the foot and the base of the walking aid. Such a sleeve could be made in a range of lengths so as to get correct strut lengths for different frame configurations.

The walking aid is attachable to any suitable leg frame, including an Ilizarov frame and an American Taylor Spatial frame.

According to a second aspect of the present invention there is provided a human or animal leg frame used for broken or fractured bones, or leg deformities, wherein the aid comprises one or more weight-bearing actuators adapted to take some or all of the user's weight when walking.

An embodiment of the present invention will herein be described by way of example only and with reference to the accompanying drawings in which;

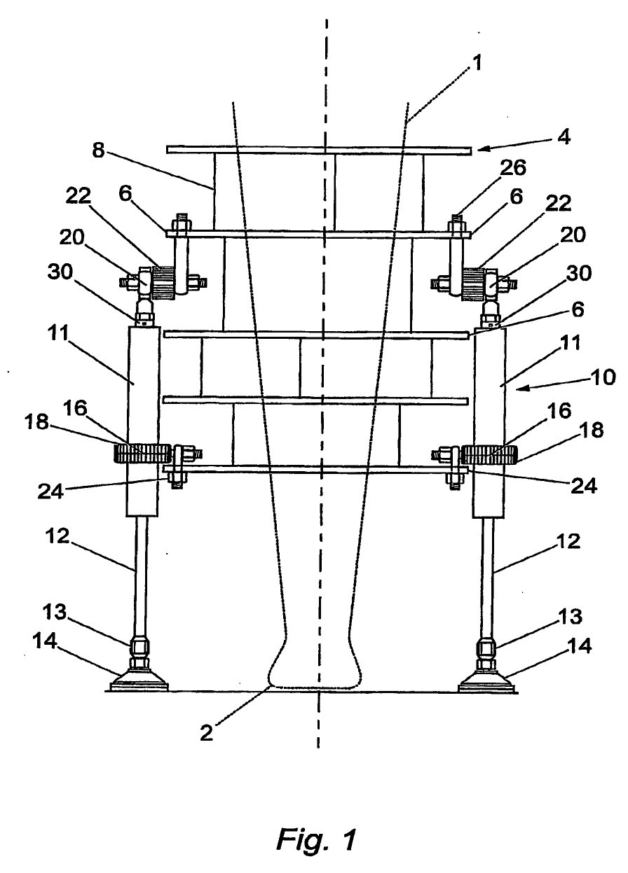

FIG. 1 as a diagrammatic front representation of a walking aid attached to an Ilizarov frame fixed to a human leg; and

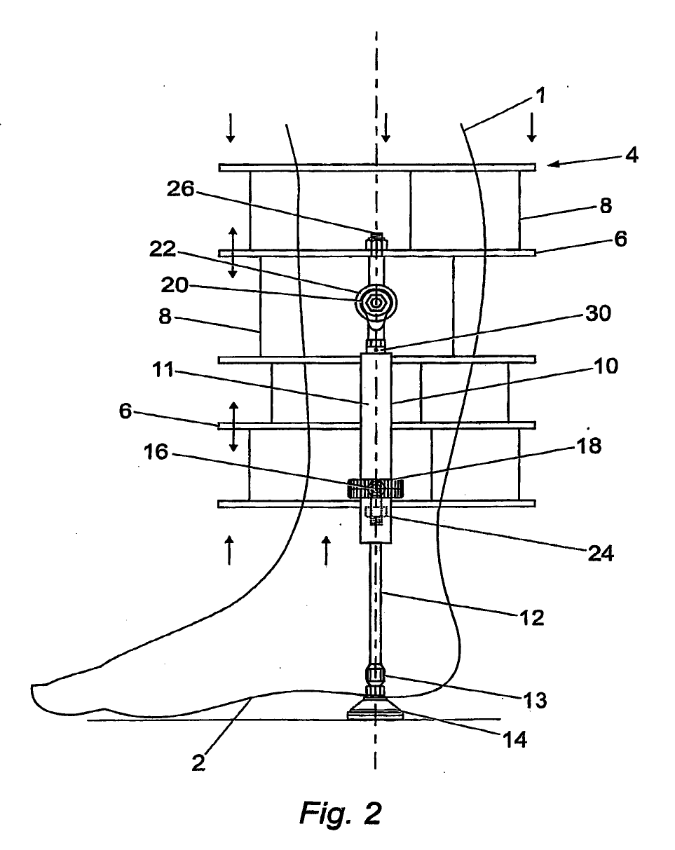

FIG. 2 is a profile view of a FIG. 1.

FIG. 1 shows a (diagrammatic) human lower leg 1 and human foot 2. A (diagrammatic) Ilizarov frame 4 is shown composed of a number of carbon fibre rings 6 that are interconnected via vertical bars 8. The frame is fixed to the human lower leg 1 via pins (not shown) that extend across the inner circumference of the carbon fibre rings 6, through the leg and the bone that is fractured or broken.

The walking aid is composed of two adjustable stainless steel gas struts 10 which each have a stainless steel housing 11, a resistibly urged piston 12, a height adjusting sleeve 13, and a stainless steel foot with a swivel base 14. The upper part of the gas strut 10 is attached to an upper carbon fibre ring 6 via outriggers at points 26. On each strut 10 is an alloy swivel 18 attached via outriggers at points 24 to a lower frame ring 6, which allow height adjustment relative to the lowest frame ring 6.

At the top of each strut 10 there is a stainless steel rose joint 20 and a rubber mount 22, which act together to allow twisting motion of the knee joint during the motion of walking.

A grub screw 30 is situated at the top of each stainless steel gas strut 10, and allows controlled release of gas from the gas strut 10.

In operation, the Ilizarov frame 4 is fixed to the lower leg 1 of the patient via pins that extend across the inner circumference of the carbon fibre rings 6 into and through skin of the leg 1 and the bone fragments that need to re-grow and re-form.

An example of steps to be followed for fitting and adjusting the struts 10 is set out below.

Weigh patient.

Get patient to push down on scales with their damaged foot to determine what weight their limb can take comfortably.

Subtract that weight from their body weight.

Divide remaining weight between the two gas struts i.e. 17 stone body weight, 6 stone on damaged limb, 11 stone remaining =5% stone on each strut.

To preset struts—push on scales with gas strut and release gas from grub screw 30 until required weight is obtained.

With patient standing hold the gas strut beside the frame to determine appropriate mounting position on frame. You may have to fit shorter or longer height adjusting sleeve 13 as needed.

Mount outrigger 26 to top position (outrigger with slot).

Mount rubber mounting 22 to outrigger 26 putting male side through and fitting nut.

Fit two-hole outrigger 24 to appropriate position at bottom of frame.

Fit alloy swivel 18 to outrigger 24.

Slide gas strut through swivel 18 and connect top to rubber mount 22 with 6 mm bolt supplied.

With patient standing, adjust struts so that strut will compress when weight is put on damaged limb.

The patient can now stand by applying weight to the injured leg 1. Some if not the majority of the patient's weight normally applied through the injured leg 1 will be born by the walking aid struts 10. When the patient stands, the stainless steel feet with swivel base 14 come in contact with the floor and bear the desired pressure according to the gas pressure set in each strut 10.

While the patient takes an initial step and lifts the injured leg 1 from the ground, the pressure is removed from the gas struts 10 so that the pistons 12 are in their generally extended position. When the patient finishes the step by again applying pressure on the foot 2 of the injured leg 1, the resistibly urged pistons 12 are pushed into their contracted position. The normal twisting action of the knee joint that occurs when the foot of a healthy individual hits the floor is allowed via the twisting motion of the rubber mounts 22. This swivel motion is further accommodated via the alloy swivels 18. The extension and contraction of the pistons 12 are controlled by hydraulics within the stainless steel housing 11.

In a modification of the gas struts, a spring or springs can be used instead of gas.

Thus, by a simple adjustment of the gas pressure within the struts of the walking aid attached to a deformed, fractured- or broken-leg frame, the patient can feel confident to apply pressure on the injured leg, as the walking aid will bear the precise amount of weight and pressure that the patient feels comfortable with. The patient can therefore walk without fearing excessive pain due to accidental over loading of the injured leg while walking or standing, which will significantly increase the rate of recovery and re-growth of the fractured or broken bone and so reduce the use of crutches, wheelchairs etc. and minimise the need for physiotherapy and other forms of recovery treatments.

1. A walking aid attachable to a human or animal leg frame, used for broken or fractured bones, or leg deformities, wherein the aid comprises at least one weight-bearing actuator adaptable to take some or all of the user's weight when walking.

2. A walking aid as claimed in claim 1 comprising at least two actuators.

3. A walking aid as claimed in claim 1 or 2 wherein the actuator(s) are piston or ram and housing arrangements.

4. A walking aid as claimed in any preceding claim wherein the actuator(s) are gas struts.

5. A walking aid as claimed in any preceding claim wherein the actuator(s) are fixable to the outside of the leg frame.

6. A walking aid as claimed in any of claims 2 to 5 wherein the actuator(s) are positioned substantially symmetrically around the frame.

7. A walking aid as claimed in any of the preceding claims wherein the actuatur(s) are dampened and/or have another controlled extension/retraction action.

8. A walking aid as claimed in claim 4 wherein the gas strut is a hydraulic gas strut.

9. A walking aid as claimed in any preceding claim wherein the aid is adjustable.

10. A walking aid as claimed in any of the preceding claims wherein the aid comprises at least one fitting chosen from the group consisting of flexible fittings and mountings such as rubber mountings, rose joints, intermediate swivel means, as well as swivel bases, involving universal joint means, to assist or allow twisting motion of the leg that normally occurs during walking.

11. A walking aid as claimed in any preceding claim which further comprises a connecting sleeve that is connectable between a foot and the base of the walking aid.

12. A walking aid as claimed in any preceding claim wherein the aid is attachable to any suitable leg frame, including an Ilizarov frame and an American Taylor Spatial frame.
